SEQUENCE LISTING (13 sequences)

SEQ ID NO: 1 - RNA, 22 nt, Homo sapiens
uauugcacuugucccggccugu

SEQ ID NO: 2 - RNA, 22 nt, Homo sapiens
uguaaacauccuacacucagcu

SEQ ID NO: 3 - RNA, 23 nt, Homo sapiens
caacggaaucccaaaagcagcug

SEQ ID NO: 4 - RNA, 22 nt, Homo sapiens
ucaggcucaguccccucccgau

SEQ ID NO: 5 - RNA, 22 nt, Homo sapiens
cuggcccucucugcccuuccgu

SEQ ID NO: 6 - RNA, 23 nt, Homo sapiens
uguaaacauccuacacucucagc

SEQ ID NO: 7 - RNA, 22 nt, Homo sapiens
uuauaauacaaccugauaagug

SEQ ID NO: 8 - RNA, 22 nt, Homo sapiens
agagguaguagguugcauaguu

SEQ ID NO: 9 - RNA, 21 nt, Homo sapiens
gccccugggccuauccuagaa

SEQ ID NO: 10 - RNA, 22 nt, Homo sapiens
uagcaccaucugaaaucgguua

SEQ ID NO: 11 - RNA, 22 nt, Homo sapiens
ucagugcacuacagaacuuugu

SEQ ID NO: 12 - RNA, 22 nt, Homo sapiens
ugucaguuugucaaauacccca

SEQ ID NO: 13 - RNA, 22 nt, Homo sapiens
cagugguuuuacccuaugguag

(19) United States
(12) Patent Application Publication
(10) Pub. No.: US 2017/0283883 A1
(43) Pub. Date: Oct. 5, 2017

(54) METHODS FOR LUNG CANCER DETECTION

(71) Applicants: FONDAZIONE ISTITUTO FIRC DI ONCOLOGIA MOLECOLARE, Milano (MI) (IT); UNIVERSITA DEGLI STUDI DI MILANO, Milano (MI) (IT); IEO - Istituto Europeo di Oncologia S.r.l., Milano (MI) (IT)

(72) Inventors: Pier Paolo Di FIORE, Milano (MI) (IT); Fabrizio BIANCHI, Milano (MI) (IT); Francesco NICASSIO, Milano (MI) (IT); Matteo Jacopo Luca Nicolo MARZI, Milano (MI) (IT); Francesca MONTANI, Milano (MI) (IT)

(21) Appl. No.: 15/509,690

(22) PCT Filed: Sep. 9, 2015
(86) PCT No.: PCT/EP2015/070664

§ 371 Date: Mar. 8, 2017

Related U.S. Application Data
(60) Provisional application No. 62/047,932, filed on Sep. 9, 2014.

(51) Int. Cl. C12Q 1/68; G06F 19/18

(52) U.S. Cl. CPC C12Q 1/6886; G06F 19/18; C12Q 2600/158; C12Q 2600/178

(57) ABSTRACT

The disclosure describes a method for diagnosing lung cancer in a subject by detecting in a biological sample obtained from that patient a miRNA signature, the presence of which provides an earlier indication of cancer than alternative art-recognized methods, including, but not limited to, low-dose computed tomography (LDCT).

### CROSS-REFERENCE TO RELATED APPLICATIONS

This application claims priority to and benefit of provisional application U.S. Ser. No. 62/047,932 filed on Sep. 9, 2014, the contents of which are incorporated herein by reference in their entirety.

### FIELD OF THE DISCLOSURE

The present disclosure relates generally to the fields of molecular biology and cancer diagnosis and therapy.

### BACKGROUND

Lung cancer is the leading cause of cancer death worldwide and its incidence continues to grow in women and in developing countries. As lung cancer is asymptomatic in its early stages, the majority of patients are diagnosed with advanced disease, for example, when the tumor is unresectable. Consequently, the overall survival rate is very low: 16% at 5 years. It is important, therefore, that screening programs and novel diagnostic tools are developed to improve the ability to detect the disease in its early stages (stage I-II) when it is still curable.

Low-dose computed tomography (LDCT) is an effective tool for the diagnosis of lung cancer, as proved by several single-arm and randomized studies. However, concerns have been raised about the feasibility and cost of a nationally implemented LDCT screening program. Although LDCT is targeted to high-risk individuals, identified by age and smoking history, a refinement of the pre-selection criteria based on additional risk factors, such as tumor markers, can prove to be of great benefit in enabling the widespread and cost-effective implementation of LDCT screening.

There is a long-felt yet unmet need for a minimally invasive and relatively cheap blood test for use as a first-line screening procedure to pre-select patients who require further diagnostic investigation by LDCT. Such a test would reduce the size of the target screening population, and would undoubtedly be advantageous in terms of costs, screening uptake rates and reduced medicalization of participants.

The disclosure provides a minimally invasive and relatively cheap blood test for use as a first-line screening procedure to pre-select patients who require further diagnostic investigation by LDCT.

### SUMMARY

The disclosure describes a method for diagnosing lung cancer in a subject by detecting in a biological sample obtained from that patient a miRNA signature, the presence of which provides an earlier indication of cancer than alternative art-recognized methods, including, but not limited to, low-dose computed tomography (LDCT).

MicroRNAs (miRNAs), short non-coding RNAs involved in the regulation of cellular differentiation, proliferation and apoptosis, are emerging as one of the most promising classes of blood-borne tumor markers. The expression of miRNAs is often deregulated in human tumors, leading to alterations in miRNA profiles in bodily fluids, including serum and plasma. Moreover, cell-free miRNAs display remarkable stability in the blood, which is attributed to the protection from harsh conditions and blood RNases provided either by microvesicles, such as exosomes, or by protein complexes, such as high-density lipoprotein complexes or argonaute proteins.

The disclosure provides evidence that a miRNA signature of detected circulating miRNAs provides an early indicator of cancer, including, lung cancer.

The disclosure provides data validating the efficacy of the methods described herein in a large-scale study aiming at detecting lung cancer, including asymptomatic and/or early-stage lung cancers in a cohort of high-risk individuals enrolled in the COSMOS (Continuous Observation of SMOking Subjects) lung cancer screening program. The data demonstrate that the methods of the disclosure provide a cost-effective, easily implementable, diagnostic screening tool for lung cancer.

The disclosure provides a method of diagnosing lung cancer in a subject in need thereof comprising (a) detecting in a biological sample from the subject a decrease in the abundance of each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p compared to a control abundance value corresponding to each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p from a control sample; (b) detecting in the biological sample an increase in the abundance of each of hsa-mir-29a-3p, hsa-mir-148a-3p, hsa-mir-223-3p, and hsa-mir-140-5p compared to a control abundance value corresponding to each of hsa-mir-29a-3p, hsa-mir-148a-3p, hsa-mir-223-3p, and hsa-mir-140-5p from a control sample; and (c) diagnosing the subject with lung cancer, when a decreased abundance in each of the miRNA in (a) is detected and an increased abundance of each of the miRNA in (b) is detected.

Methods of the disclosure may be used to diagnose asymptomatic lung cancer and/or early stage lung cancer. Methods of the disclosure may be used to diagnose any subtype of lung cancer.

Biological samples and/or control biological samples of the methods of the disclosure may comprise, consist essentially of or consist of a biological fluid. Exemplary biological fluids include, but are not limited to, saliva, urine, blood, or lymph fluid. Biological samples and/or control biological samples of the methods of the disclosure may comprise, consist essentially of or consist of blood, whole blood, blood plasma and/or blood serum. Biological samples of the methods of the disclosure may comprise, consist essentially of or consist of blood serum.

Control biological samples of the methods of the disclosure may comprise, consist essentially of or consist of blood, whole blood, blood plasma and/or blood serum obtained from at least two normal subjects. Control biological samples of the methods of the disclosure may comprise, consist essentially of or consist of blood serum obtained from at least two normal subjects.

According to the methods of the disclosure, a decrease in the abundance of each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p compared to a control abundance value may be a statistically significant difference from the control value. Alternatively, or in addition, an increase in the abundance of each of hsa-mir-29a-3p, hsa-mir-148a-3p, hsa-mir-223-3p, and hsa-mir-140-5p compared to a control abundance value may be a statistically significant difference from the control value.

Statistically significant differences of the disclosure may be characterized by a p-value of less than 0.05. Statistically significant differences of the disclosure may be characterized by a p-value of less than 0.001. Statistically significant differences of the disclosure may be characterized by a p-value of less than 0.0001.

According to the methods of the disclosure, the decrease in the abundance of each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p compared to a control abundance value may be expressed as a fold-difference. Alternatively, or in addition, an increase in the abundance of each of hsa-mir-29a-3p, hsa-mir-148a-3p, hsa-mir-223-3p, and hsa-mir-140-5p compared to a control abundance value may be expressed as a fold-difference.

The disclosure provides a method of diagnosing lung cancer in a subject in need thereof comprising (a) detecting in a biological sample from the subject a decrease in the abundance of each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p compared to a control abundance value corresponding to each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p from a control sample; (b) detecting in the biological sample an increase in the abundance of each of hsa-mir-29a-3p, hsa-mir-148a-3p, hsa-mir-223-3p, and hsa-mir-140-5p compared to a control abundance value corresponding to each of hsa-mir-29a-3p, hsa-mir-148a-3p, hsa-mir-223-3p, and hsa-mir-140-5p from a control sample; (c) calculating a risk score and (d) diagnosing the subject with lung cancer, when a decreased abundance in each of the miRNA in (a) is detected and an increased abundance of each of the miRNA in (b) is detected. In certain embodiments of this method, the risk score is a product of (a) a fold decrease and a weight coefficient, or (b) a fold increase and a weight coefficient, wherein the weight coefficient is determined by, for example, a diagonal linear discriminant analysis (DLDA).

Control values for each miRNA of the methods of the disclosure may be determined by a method comprising detecting in a control biological sample from a normal subject an abundance of each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, hsa-mir-331-3p, hsa-mir-29a-3p, hsa-mir-148a-3p, hsa-mir-223-3p, and hsa-mir-140-5p. In certain embodiments of these methods, the at least one normal subject of the disclosure does not have cancer. Alternatively, or in addition, the at least one normal subject does not have lung cancer.

Subjects of the methods of the disclosure may be male or female. Subjects of the methods of the disclosure may be any age; however, subjects are preferably adults.

Subjects of the methods of the disclosure may be asymptomatic.

Subjects of the methods of the disclosure may have stage I or stage II lung cancer.

Subjects of the methods of the disclosure may present one or more risk factors for developing lung cancer. Exemplary risk factors for developing lung cancer include, but are not limited to, a personal or family history of cancer, a history of smoking and/or exposure to second-hand smoke, and/or having limited access to preventative or curative medical care. Alternatively, or in addition, a subject who is exposed to fine particulates from his or her environment is at an increased risk of developing lung cancer. For example, an individual who is exposed to smoke (including secondhand smoke), radon gas, asbestos and other chemicals (e.g. arsenic, chromium, and/or nickel), and/or nano-size particulates (e.g. dust and particulates from a manufacturing facility and/or motor vehicle exhaust) are at an increased risk of developing lung cancer. The combination of a family history of cancer and any one or more of the risk factors described herein may further increase an individual's risk of developing lung cancer.

Methods of the disclosure may further comprise the step of performing low-dose computed tomography (LDCT) or referring the subject for LDCT if the subject is diagnosed with lung cancer.

Methods of the disclosure may further comprise the step of providing a treatment or referring the subject for treatment if the subject is diagnosed with lung cancer.

According to the certain embodiments of the methods of the disclosure, detecting step of (a) and/or (b) may comprise transcribing each of the miRNA in steps (a) and/or (b) using at least one human miRNA stem-loop primer specific for at least one miRNA of (a) or (b) to generate a complementary DNA (cDNA) corresponding to each of the miRNA, amplifying each of the cDNA, and determining a relative abundance of each of the cDNA compared to at least one housekeeping miRNA. In certain embodiments of these methods, the at least one human miRNA stem-loop primer specific for at least one miRNA and the at least one miRNA hybridize to form at least one duplex. In certain embodiments of these methods, the amplifying step comprises performing quantitative real-time polymerase chain reaction (qRT-PCR). In certain embodiments of these methods, the at least two housekeeping miRNA comprise miR-197, miR-19b, miR-24, miR-146, miR-15b, or miR-19a. Alternatively, the at least two housekeeping miRNA may comprise miR-197, miR-19b, miR-24, miR-146, miR-15b and miR-19a.

In certain embodiments of these methods, the relative abundance of each of the cDNA is determined by adding a scaling factor to the raw cycle threshold (CT) of each of the cDNA to generate a normalized (CT) value. Scaling factors of the disclosure may be determined by determining a mean cycle threshold of the at least two housekeeping miRNA selected from the group consisting of miR-197, miR-19b, miR-24, miR-146, miR-15b, and miR-19a and subtracting the mean CT from a constant value (K). In certain embodiments of the methods of the disclosure, the constant value (K) equals 21.646. In certain embodiments of these methods, the normalized CT value is used to determine a weight coefficient.

Methods of the disclosure may further comprise the step of determining a risk score for the subject. In certain embodiments of these methods, risk score is calculated according to: RS=−(Σᵢwᵢ xᵢ−THRES), wherein i corresponds to each of the miRNAs of (a) and (b), respectively, and wherein THRES=−261.779, wᵢ is the weight coefficient of the i^{th} miRNA, and xᵢ is the expression value of the i^{th} miRNA. The expression value of the i^{th} miRNA may also be referred to as the raw CT of the i^{th} miRNA. In certain embodiments of these methods, the risk score is greater than or equal to 5, indicating that the subject has a high risk of developing cancer. In certain embodiments of these methods, the risk score is less than 5 and greater than or equal to −5, indicating that the subject has an intermediate risk of developing cancer. In certain embodiments of these methods, the risk score is less than −5, indicating that the subject has a low high risk of developing cancer.

### BRIEF DESCRIPTION OF THE DRAWINGS

The drawings are primarily for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a graph that depicts the Study design. Sera were obtained from two independent collections: (i) the COSMOS study [48 patients with lung cancer (T), 984 (12+972) individuals selected from a consecutive cohort without lung cancer (N), 38 patients with benign pulmonary nodules (NOD), 16 patients with chronic obstructive pulmonary disease (COPD), 24 patients with pneumonia (PN), and 5 patients who were operated for suspected lung cancer, but were negative at histological analysis, i.e. surgical false positives (Benign)]; (ii) the Division of Thoracic Surgery of the European Institute of Oncology (IEO) [74 patients with lung cancer (T)]. Sera samples from the COSMOS study were divided into a Calibration Set, a Validation Set and a Specificity Set (see Example 1). Sera samples from Thoracic Surgery comprised the Clinical set.

FIG. 2A is a pair of graphs that depict the receiver operating characteristic (ROC) curves of the miR-Test in the Validation set. Abbreviations: AUC, area under curve; T, patients with lung cancer, N, individuals without lung cancer).

FIG. 2B is a pair of graphs that depict the miR-Test risk scores in the Specificity set plus COSMOS lung cancer patients (left panel) and in the Clinical set (right panel). Average risk scores and p-values (one-way ANOVA) are also shown. Dashed line indicates miR-Test cutoff (=0) used to decide positive or negative results in the two-category stratification (NOD, patients with LDCT-detected, non-calcified, lung nodules stable in size at >5 years of follow-up; COPD, patients with chronic obstructive pulmonary disease; PN, patients with lung pneumonia; Benign, patients operated for suspected lung cancer, but negative at histological analysis (surgical false positives); Tumor, lung cancer patients from COSMOS trial: AC, adenocarcinoma; SCC, squamous cell carcinoma; LCC, large cell carcinoma; LCC, large cell lung cancer; SCLC, small cell lung cancer. Number of patients in groups is shown in parentheses.

FIG. 2C is a graph that depicts the miR-Test risk scores for lung cancer patients from the Clinical set with the same tumor histological subtype (i.e. adenocarcinoma), stratified for different smoking status. Average risk scores and p-values (one-way ANOVA) are also shown (Ex-smokers, patients who quit smoking >5 years before lung cancer diagnosis).

FIG. 3 is a pair of graphs that depict the performance of miR-Test after surgical removal of the tumor. MiR-Test risk scores of a set of 16 patients with stage I non-small cell lung cancer (NSCLC) for whom serum samples were collected before and after surgery at 1, 5 and 12 months during follow-up visits. The number of patients varies depending on the availability of matched serum samples and is indicated at the bottom of the line graphs (Risk, miR-Test risk score; pts, patients; pre-, pre-surgery; post-, post-surgery; mo, months post-surgery). P-values (P) were calculated using one tailed paired t-test. Dashed line indicates miR-Test cutoff (=0) used to assign a positive or negative test result.

FIG. 4A is a pair of graphs that depict the unsupervised clustering analysis of the 147 known circulating miRNAs (147-miRNA) in the NCI-60 cell line panel dataset. Normalized NCI-60 miRNA expression profile data (OSU V3 chip) were downloaded using the CellMiner web application (version 1.5; [30]). Probeset-level data were mean centered before clustering. Grayscale bar indicates miRNA log 2 relative expression (Type′, tissue of origin of derived cell lines: LC, non-small cell lung cancer; LE, leukemia; OV, ovarian cancer; BC, breast cancer; RE, renal cancer; CNS, tumors of central nervous system; CO, colon cancer; PR, prostate cancer. ‘Cluster Type’, clusters were defined following main tree branches: Epith-like (epithelial-like), miRNAs preferentially expressed in LC cells; Inflam-like (inflammatory-like), miRNAs preferentially expressed in leukemic cells; Undefined, miRNAs whose expression is heterogeneous in LC and LE cells).

FIG. 4B is a pair of graphs that depict the unsupervised clustering analysis of the 34- and 13-miRNA signatures restricted to NSCLC or leukemic NCI-60 cell lines. Probeset-level data were mean centered before clustering.

FIG. 4C is a pair of bar plots of quantities of the “inflammatory-like” and “epithelial-like” miRNA components of the 34-miRNA signature, in the serum of patients with or without lung cancer from the Validation set. Grayscale indicates different miRNAs. In bold, miRNA present also in the 13-miRNA signature (miR-Test). Asterisks indicate significantly different miRNA serum quantities between lung cancer patients and healthy individuals (p<0.05). P-values were calculated by Student's t-test.

FIG. 4D is a pair of bar plots of quantities of the “inflammatory-like” and “epithelial-like” miRNA components in miR-Test positive individuals selected from the Validation and Specificity sets (Healthy, individuals without lung nodules or other lung diseases from the 972 COSMOS cohort (False positives; N=18); NMD, patients with non-malignant lung disease from the Specificity set (False positives; N=11); Tumor, lung cancer patients from the COSMOS cohort (N=28)). Y-axes, -ddCT of qRT-PCR data). Asterisks, statistically significant differences in miRNA serum quantities between the NMD or Tumor patient groups compared to healthy individuals (p<0.05). ^{§} Statistically significant differences in miRNA serum quantities in lung cancer patients compared to healthy individuals or to NMD patients (Tumor specific, p<0.05). P-values were calculated by Student's t-test.

FIG. 5A is a graph that depicts the lung cancer mortality stratified by miR-29a serum quantities. A competing risk approach was applied to estimate the cumulative incidence of lung cancer mortality. Gray's Test was performed to test differences in stratification (miR-29a High, serum quantities higher than the median value (Ct≦26.5); miR-29a Low, serum quantities below the median value (Ct>26.5)).

FIG. 5B is a graph that depicts the serum quantities of miR-29a analyzed by qRT-PCR in lung cancer patients and patients operated with benign disease (surgery false positives). Y-axes, CT normalized to the six housekeeping miRNAs (see Example 1). P-values were calculated by Welch's t-test.

FIG. 6 is a series of correlation plots of Risk scores obtained using the original signature (34-miRNA) or the ‘reduced’ 13-miRNA signature in all cohorts of patients analyzed in the study. Pearson's correlation coefficients were calculated using JMP software. The assigned risk scores from the two models (34- or 13-miRNA) strongly correlated (Pearson's r≧0.96) in all cohorts, supporting the reliability of the 13-miRNA model (miR-Test).

FIG. 7 is a graph that depicts the distribution of 13-miRNA Risk scores centered on their mean of 36 replicate analyses of the same serum sample. Dashed lines indicate 1 standard deviation from the mean (±σ).

### DETAILED DESCRIPTION

The disclosure provides a “miR-Test” for the early detection of lung cancer. An exemplary embodiment of the miR-test comprises a method of diagnosing a subject with lung cancer, including asymptomatic and/or early stage lung cancer, comprising (a) obtaining a biological sample from the subject; (b) detecting in the biological sample a decrease in the abundance of each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p compared to a control abundance value corresponding to each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p; and (c) detecting in the biological sample an increase in the abundance of each of hsa-mir-29a-3p, hsa-mir-148a-3p, hsa-mir-223-3p, and hsa-mir-140-5p compared to a control abundance value corresponding to each of hsa-mir-29a-3p, hsa-mir-148a-3p, hsa-mir-223-3p, and hsa-mir-140-5p; wherein the detection of a decreased abundance in each of the miRNA in (b) and an increased abundance of each of the miRNA in (c) indicates the development of lung cancer in the subject, thereby diagnosing the subject with lung cancer. In support of the miR-Test of the disclosure, data are provided for the validation of this method of diagnosing lung cancer, including asymptomatic and/or early stage lung cancer, in a large cohort of high-risk individuals. In this study, the test reached a sensitivity of 86%, when the high and intermediate risk classes were grouped together, while 53% of individuals were positioned in the low-miRNA risk category. The NPV of the miR-Test was >99%, thus, low-risk individuals can safely avoid subsequent LDCT screening. The high sensitivity and NPV obtained with the miR-Test are comparable to those observed with LDCT alone, which indicates that the miR-Test could substitute the LDCT as a first-line screening tool. Conversely, the lower specificity of the test compared to LDCT, would not affect the overall screening result, as cases with a positive miR-Test result would be required to undergo LDCT to confirm diagnosis and localize neoplastic lesions for subsequent surgery.

With respect to the false negative (i.e. miR-Test-negative lung cancer patients) and the false positive (miR-Test-positive individuals, but negative upon LDCT) results, only 2 out of 15 deaths (Validation and Clinical Sets) were false negatives. The death rate per 1000 patients/year was 0, 51, and 71, in the low, intermediate and high-risk categories, respectively. While the relatively low number of deaths in our cohort limits the statistical power of these results, it is plausible that tumors missed by the miR-Test may be rather indolent or even represent overdiagnosis by LDCT. Notably, 5 of 5 patients with benign tumors, who had a positive LDCT result, were miR-Test-negative. Similarly, most individuals with NMD (72 out of 83; 87%) were miR-Test-negative. This latter result is relevant since in LDCT screening trials there is a high rate of false-positive findings, up to 28%. This complicates the interpretation of LDCT results and ensuing decisions about the screening time interval: a difficulty that might be alleviated by a first-line screening test, such as the miR-Test, that significantly reduces unnecessary LDCTs for individuals without lung cancer. With respect to miR-Test false positives, these events may not necessarily represent an intrinsic limitation of the test, because it is possible that the blood test can anticipate the LDCT diagnosis.

The miR-Test possesses the characteristics of accuracy and robustness required to be introduced as a first-line tool in lung cancer screening programs. If implemented, the miR-Test would result in a reduction in the number of LDCTs by more than 50%, while retaining the diagnostic sensitivity of LDCT.

### EXAMPLES

### Example 1: miRNA Blood Test Study

### Study Population

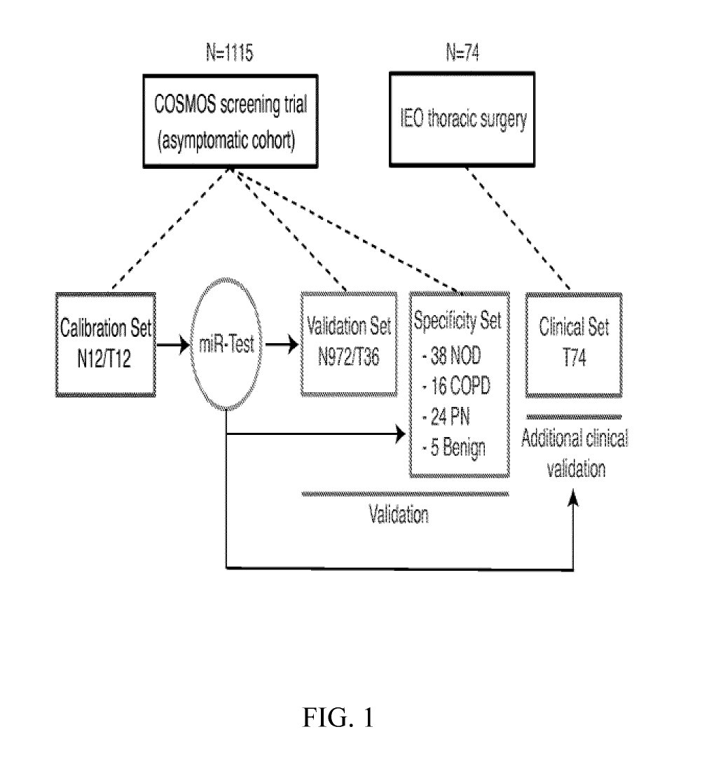

Four independent cohorts of patients and individuals were employed in the study: i) Calibration set, ii) Validation set, iii) Specificity set, iv) Clinical set (see FIG. 1):

Calibration Set:

Twenty-four individuals were selected from the COSMOS trial (12 with screen-detected lung cancer and 12 lung cancer free; FIG. 1, Table 2) and used to refine the miRNA signature (i.e. the miR-Test). The 12 lung cancer patients were previously screened to derive the 34-miRNA signature (see, Vickers K C, Palmisano B T, Shoucri B M, et al. Nat Cell Biol 2011; 13(4):423-33).

Validation Set:

The miR-Test was validated in an independent set of 1008 individuals enrolled in the COSMOS study including 36 patients with LDCT detected lung cancer and 972 individuals without lung cancer, randomly selected from a consecutive cohort from March 2011 to March 2012.

Specificity Set:

A third cohort of 83 patients was used for further validation of the miR-Test. These individuals were selected from COSMOS study participants and were not included in any of the other sets used in this study. This cohort was composed of: i) 38 individuals with CT-detected solitary pulmonary nodules stable in size at 5 years of follow-up; ii) 16 patients with chronic obstructive pulmonary disease; iii) 24 individuals with pneumonia; iv) and 5 with operated benign lung tumor. Importantly, none of these individuals developed lung cancer during a >5-year follow-up period by LDCT.

Clinical Set:

A fourth independent cohort of 74 patients diagnosed with lung cancer outside of the COSMOS trial was used. These patients underwent surgery at the European Institute of Oncology from November 2005 to January 2008.

Information about the clinical and pathological characteristics for all individuals and patients screened by the miR-Test is reported in Table 1 and Table 2. The mean±standard deviation (SD) is reported for age (years) and smoking status (pack-years), and the median, interquartile (Q1;Q3) and overall range (min-max) is reported for age. The information on pack-years was available for nearly all individuals except for: 25 patients (a) and 36 patients (h) (AC, lung adenocarcinoma; SCC, lung squamous cell carcinoma; SCLC, small cell lung cancer; LCC, large cell lung cancer; COPD, chronic obstructive pulmonary disease; NOD, stable solitary pulmonary nodules; PN, pneumonia; Benign, patients operated with benign pulmonary nodules (surgery false positives)). Tumor stage was defined based on the TNM Classification of Malignant Tumors published by the International Union Against Cancer (UICC), 7th Edition. Percentages could not add up to 100 due to rounding.

TABLE 2
Clinicopathological features of the Calibration Set used to refine
the circulating miRNA signature.

| | Calibration set | | | | | |
|---|---|---|---|---|---|---|
| | All | | Tumor | | Normal | |
| | (N = 24) | | (N = 12) | | (N = 12) | |
| | N | % | N | % | N | % |
| Gender | | | | | | |
| Female | 6 | 25.0 | 3 | 25.0 | 3 | 25.0 |
| Male | 18 | 75.0 | 9 | 75.0 | 9 | 75.00 |
| Age [years] | | | | | | |
| mean ± SD | 55 ± 2 | | 55 ± 2 | | 55 ± 2 | |
| median (Q1; Q3) | 55 (53; 57) | | 55 (53; 57) | | 54 (53; 57) | |
| min-max | 51-59 | | 51-59 | | 51-59 | |
| Smoking status | | | | | | |
| Current, ex-smoker | 24 | 100.0 | 12 | 100.0 | 12 | 100.0 |
| Pack-years means ± SD | 57 ± 21 | | 55 ± 22 | | 58 ± 21 | |
| Nodule type | | | | | | |
| No Nodule | 12 | 50.0 | 0 | 0.0 | 12 | 100.0 |
| Nodule size [mm] | | | | | | |
| <5 | 0 | 0.0 | 0 | 0.0 | — | |
| 5-8 | 5 | 41.7 | 5 | 41.7 | | |
| >8 | 7 | 58.3 | 7 | 58.3 | | |
| Tumor subtype | | | | | | |
| AC | 12 | 100.0 | 12 | 100.0 | — | |
| SCC | 0 | 0.0 | 0 | 0.0 | | |
| SCLC/LCC | 0 | 0.0 | 0 | 0.0 | | |
| Tumor Stage | | | | | | |
| IA | 8 | 66.7 | 8 | 66.7 | — | |
| IIB | 3 | 25.0 | 3 | 25.0 | | |
| IIA | 0 | 0.0 | 0 | 0.0 | | |
| IIB | 0 | 0.0 | 0 | 0.0 | | |
| IIIA | 1 | 8.3 | 1 | 8.3 | | |
| IIIB | 0 | 0.0 | 0 | 0.0 | | |

The 12 asymptomatic lung cancer patients in the ‘calibration-set’ were part of the original cohort of 174 individuals used to derive the miR-Test.

Tumor stage at the time of diagnosis was determined according to guidelines of the American Joint Committee on Cancer (http://www.cancerstaging.org/). Informed consent was obtained from all participants. Patients and individuals of the Calibration, Validation and Specificity sets were all enrolled in the COSMOS study, a screening program for the anticipation of lung cancer diagnosis in high-risk individuals. All enrolled individuals were smokers or former smokers with a smoking exposure of more than 20 pack-years, aged over 50.

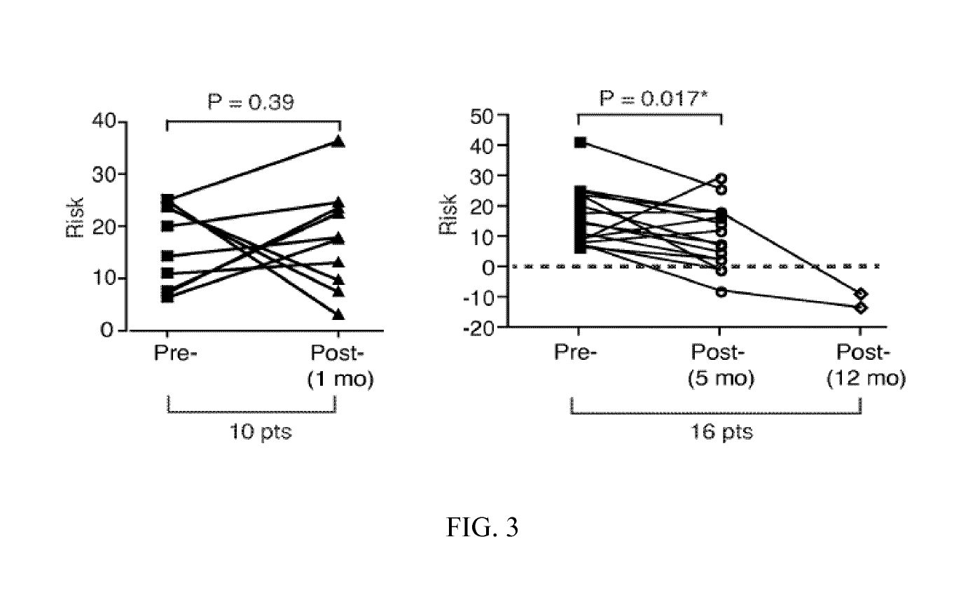

For the experiments shown in FIG. 3, sera were collected before and after surgery from a group of lung cancer patients with Stage I disease that did not receive chemotherapy.

### Blood Collection

Blood samples (10 mL) were collected by standard phlebotomy before any analysis or instrumental procedure. The first 3 mL of blood were not used for serum preparation to prevent contamination by skin. Serum was prepared by collecting blood in tubes with clot activator (S-Monovette 7.5 ml, REF01.1601—Sarstedt), left at room temperature for 3 hours to clot, then spun at 3000 rpm (1000 g, Megafuge 2.0 Heraeus) for 10 minutes at RT. The serum was removed immediately after centrifugation, leaving a 0.5 cm leftover to avoid disturbing the serum-clot interface. Serum was then aliquoted in barcoded cryotubes and snap frozen in dry ice. Aliquots were stored in a dedicated −80° C. freezer.

Serum miRNA Purification and Expression Profiling

Total RNA purification, including miRNAs, was based on lysis with Guanidinium thiocyanate-phenol-chloroform extraction (TriZol-LS, Applied Biosystem) and Spin Column-based total RNA purification (MiRneasy Mini Kit, Qiagen). Briefly, 0.3 mL of serum were mixed with Trizol-LS in volumetric ratios of 3:1 for lysis. After denaturation, a spike of synthetic miRNA (5×10⁸ copies of miR-34a) was added to the solution to monitor the extraction efficiency. A volume of 0.24 mL of Chloroform was added to the solution, which was then centrifuged for 15 min at 11,800 g at 4° C. A fixed amount (0.35 mL; around 70% of the final volume of the sample) of aqueous phase was recovered. This choice was preferred to limit contamination from the interphase.

The subsequent steps were performed in automation through a Qiacube machine (QIAGEN), according to the “miRNEASY Mini” standard protocol. RNA was eluted in 30 mL and stored at −80° C. until further analysis. Reverse transcription (RT) and pre-amplification reaction (Pre-AMP) protocols were optimized by using a MicroLab Star Liquid Handling. RT was performed from a fixed volume (3 μL) of total RNA with TaqMan MicroRNA Reverse Transcription Kit and a custom pool of Human miRNA-specific stem-loop primers (Applied Biosystem) according to the manufacturer's instruction, in a 2720 Thermo Cycler (Applied Biosystem). RT product was then diluted 1:2 with water; 5 μL of diluted RT product were pre-amplified (12 cycles of PCR) with TaqMan PreAmp Master Mix (2×) and Megaplex PreAmp Primers Human PoolA (Applied Biosystem), according to the manufacturer's instruction. The pre-amplification reaction was diluted 1:4 with Tris-EDTA buffer 0.1×. Next, 6 μL of diluted pre-amplification reaction were combined with 46.5 μL of water and with 52.5 μL of TaqMan Universal Master Mix II (Applied Biosystem). The final solution (105 μL) was loaded into one lane of a custom TaqMan®Low Density Array microRNA Custom Panel (Applied Biosystem). qRT-PCR was carried out on an Applied Biosystems 7900HT thermocycler using the manufacturer's recommended cycling conditions. A Hamilton MICROLAB® STAR liquid handling workstation was used to automate most of the described processes.

qRT-PCR Data Analysis—miRNA Profiling and Data Normalization

Data were exported into text format for further analysis. If qRT-PCR amplification curves presented a “Tholdfail” error flag (i.e. the automatic thresholding algorithm failed), data were discarded. Subsequently, raw Ct values were normalized.

To run the miR-Test, data were normalized using mean CTs (HK-mean) of six “housekeeping” miRNAs genes (miR-197, miR-19b, miR-24, miR-146, miR-15b, miR-19a) using the following procedure: a scaling factor (SF) was calculated for each sample by subtracting the “HK-mean” to a constant value (K=21.646). Data were then normalized by these SFs to eliminate technical fluctuations, using the formula:

SF+miRNA CT_{raw}=CT_{normalized} (1)

If CT raw was greater than 30.01 or ‘undetermined’ by qRT-PCR, data was set to 30.01 and normalization skipped. This normalization strategy assures an independency from the original set of samples used to train the miR-Test, and allow new samples classification without the need of an internal reference.

The calibration set was used to: i) refine diagonal linear discriminant analysis (DLDA) weight coefficients (wᵢ) of the miRNA signature by using DLDA as class metric (BRB-ArrayTools version 4.3.0—Beta_2 Release); ii) reduce the number of miRNAs in the signature. The leave-one-out (LOO) cross-validation procedure was used to cross-validate the classifier and estimate sensitivity and specificity in the calibration set. Parametric t-test set at 0.05 significance level was used for feature selection. Statistical significance of the performance of the classifier was assessed by performing 1000 permutations of class labels. Samples were then classified as positive or negative if the absolute value of the inner sum of weighted expressions (CTₙₒᵣₘ*wᵢ) was greater than the absolute value of the threshold. Since both weights and threshold are negative, the risk score was calculated as:

RS=(Σᵢwᵢxᵢ−THRES), for i=1 . . . 13 (13-miRNA signature) (2)

where THRES=−261.779, wᵢ is the weight coefficient of the i^{th} miRNA (Table 3), and xᵢ is the expression value (CT) of the i^{th} miRNA.

All analyses were automated using a custom R script developed in-house using the R statistical software version 2.14.1. The script allows to read the information content of the output documents from the Applied Biosystems ViiA 7™ and provides miR-Test risk scores automatically.

miRNA Signature Refinement

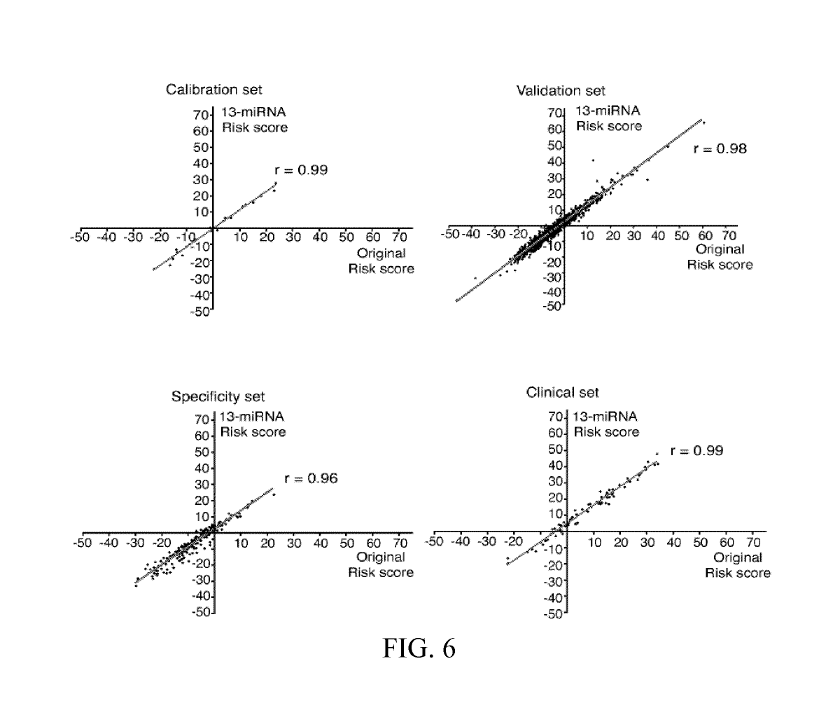

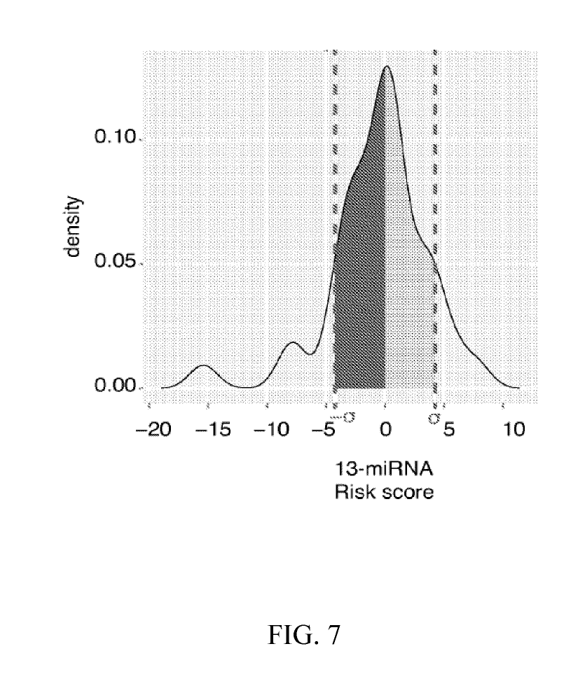

The Calibration set was used to refine a 34-miRNA signature (see, Bianchi F, Nicassio F, Marzi M, et al. EMBO molecular medicine 2011; 3(8):495-503) by employing an optimized serum miRNA detection protocol. Briefly, a pre-amplification step (PreAMP) that improved the detection of circulating miRNAs [˜23 versus ˜31 cycle threshold (Ct), on average] was added, and all steps for purification and sample preparation (including PreAMP) were automated. This procedure minimized technical variability that resulted in a lower number of miRNAs in the signature from 34 to 13, while preserving the performance of the original model (Tables 3 and 4; FIG. 6). The 13-miRNA signature (miR-Test) displayed a ±5 fluctuation of miR-Test scores when repeated measurements of the same sample were performed (FIG. 7). Based upon these results, three categories of risk (i.e., high, intermediate and low) were defined as follows: high risk scores correspond to values≧5; intermediate risk scores correspond to values<5 and ≧−5; low risk scores correspond to values<−5.

Table 3 reports the miRNA assay, accession numbers (Acc.), sequence, and miRbase nomenclature (release 20) for the 13 miRNAs that comprise the miR-Test. Fold-change (Fold) and p-values (parametric t-test) refer to the expression of miRNAs in the 12 tumor sera versus the 12 normal sera of the Calibration set. Wᵢ is the weight coefficient computed by diagonal linear discriminant analysis (DLDA) and used in the miR-Test to compute risk scores.

TABLE 3
The 13 miRNAs that comprise the miR-Test (from top to bottom SEQ ID Nos: 1-13, respectively).

| | Acc. | Acc. | | miRbase | | P- | |
|---|---|---|---|---|---|---|---|
| Assay | pre-miRNA | mature-miRNA | Sequence | ID | Fold | value | Wi |
| hsa-mir-92a-000431 | MI0000094 | MIMAT0000092 | UAUUGCACUUGUCCCGGCCUGU | hsa-miR- | -1.67 | 0.001 | -3.72 |
| | | | | 92a-3p | | | |
| hsa-mir-30b-000602 | MI0000441 | MIMAT0000420 | UGUAAACAUCCUACACUCAGCU | hsa-mir- | -1.49 | 0.003 | -3.32 |
| | | | | 30b-5p | | | |
| hsa-mir-191-002299 | MI0000465 | MIMAT0000440 | CAACGGAAUCCCAAAAGCAGCUG | hsa-mir- | -1.37 | 0.004 | -3.77 |
| | | | | 191-5p | | | |
| hsa-mir-484-001821 | MI0002468 | MIMAT0002174 | UCAGGUCAGUCCCCUCCCGAU | hsa-mir- | -1.37 | 0.010 | -2.94 |
| | | | | 484 | | | |
| hsa-mir-328-000543 | MI0000804 | MIMAT0000752 | CUGGCCCUCUCUGCCCUUCCGU | hsa-mir- | -1.30 | 0.018 | -2.92 |
| | | | | 328-3p | | | |
| hsa-mir-30c-000419 | MI0000736 | MIMAT0000244 | UGUAAACAUCCUACACUCUCAGC | hsa-mir- | -1.35 | 0.026 | -2.22 |
| | | | | 30c-5p | | | |
| hsa-mir-374a-000563 | MI0000782 | MIMAT0000727 | UUAUAAUACAACCUGAUAAGUG | hsa-miR- | -1.49 | 0.032 | -1.52 |
| | | | | 374a-5p | | | |
| hsa-let-7d-002283 | MI0000065 | MIMAT0000065 | AGAGGUAGUAGGUUGCAUAGUU | hsa-let- | -1.35 | 0.040 | -1.80 |
| | | | | 7d-5p | | | |
| hsa-mir-331-3p-000545 | MI0000812 | MIMAT0000760 | GCCCCUGGGCCUAUCCUAGAA | hsa-mir- | -1.45 | 0.049 | -1.33 |
| | | | | 331-3p | | | |
| hsa-mir-29a-002112 | MI0000087 | MIMAT0000086 | UAGCACCAUCUGAAAUCGGUUA | hsa-mir- | 1.68 | 0.013 | 1.62 |
| | | | | 29a-3p | | | |
| hsa-mir-148a4-000470 | MI0000253 | MIMAT0000253 | UCAGUGCACUACAGAACUUUGU | hsa-mir- | 1.80 | 0.003 | 2.12 |
| | | | | 148a-3p | | | |
| hsa-mir-223-002295 | MI0000300 | MIMAT0000280 | UGUCAGUUUGUCAAAUACCCCA | hsa-mir- | 1.85 | 0.003 | 2.19 |
| | | | | 223-3p | | | |
| hsa-mir-140-5p-001187 | MI0000456 | MIMAT0000431 | CAGUGGUUUUACCCUAUGGUAG | hsa-mir- | 1.92 | <0.0001 | 5.38 |
| | | | | 140-5p | | | |

The sensitivity (SEN) and specificity (SPE) of the original 34-miRNA signature and the reduced 13-miRNA signature (the miR-Test) in the Calibration Set is depicted in Table 4. Sensitivity and specificity are based on cross-validation results of the diagonal linear discriminant analysis (DLDA) classifier (see Example 1). A risk score of 0 was used to compute sensitivity and specificity.

TABLE 4
Comparison of the performance of the 34- and 13-miRNA
signatures in the Calibration Set.

| | SEN | SPE |
|---|---|---|
| | % | % |
| 34-miRNA model | 75 | 75 |
| 13-miRNA model | 75 | 83 |

### Statistical Analyses

Analyses were performed using JMP 10 software (SAS) and SAS 9.3 (SAS Institute, Cary N.C.). Cumulative lung cancer-specific mortality was estimated through the competing risks approach.

### Example 2: Validation of the miR-Test in a Lung Cancer Screening Program

A multi-tiered study was designed on high-risk individuals (heavy smokers, aged>50) enrolled in the lung cancer screening trial COSMOS, and on lung cancer patients diagnosed outside of screening (FIG. 1). In an initial step, the original 34-miRNA signature was refined (see, Bianchi et al), taking into consideration a number of technological improvements (see Example 1). This refinement allowed a reduction of the signature to 13 miRNAs (henceforth, the miR-Test, Table 2 and 3), which maintained the same performance as the original 34-miRNA signature (Table 4, and FIG. 6). The reduction of the signature is advantageous in terms of translation into clinical practice, as it reduces the costs and complexity of the test.

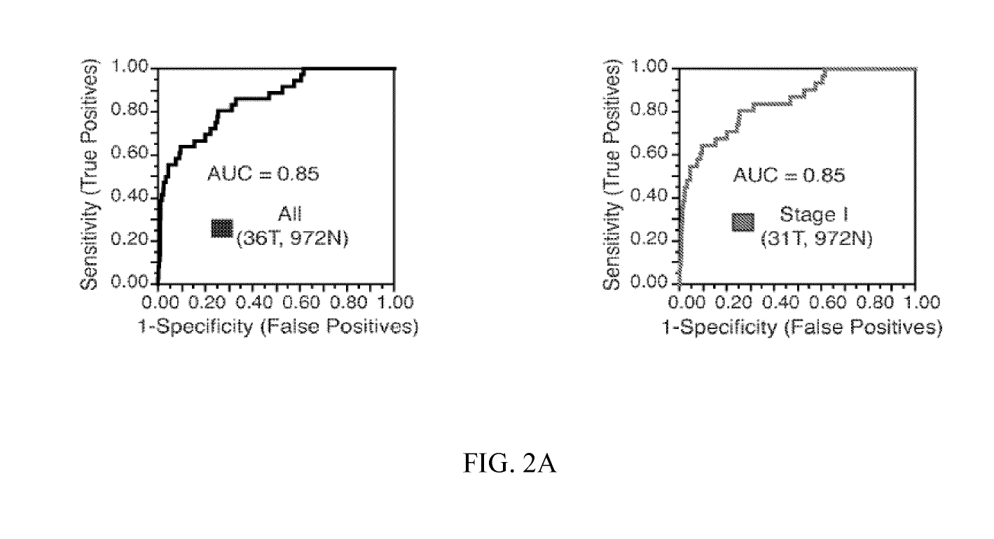

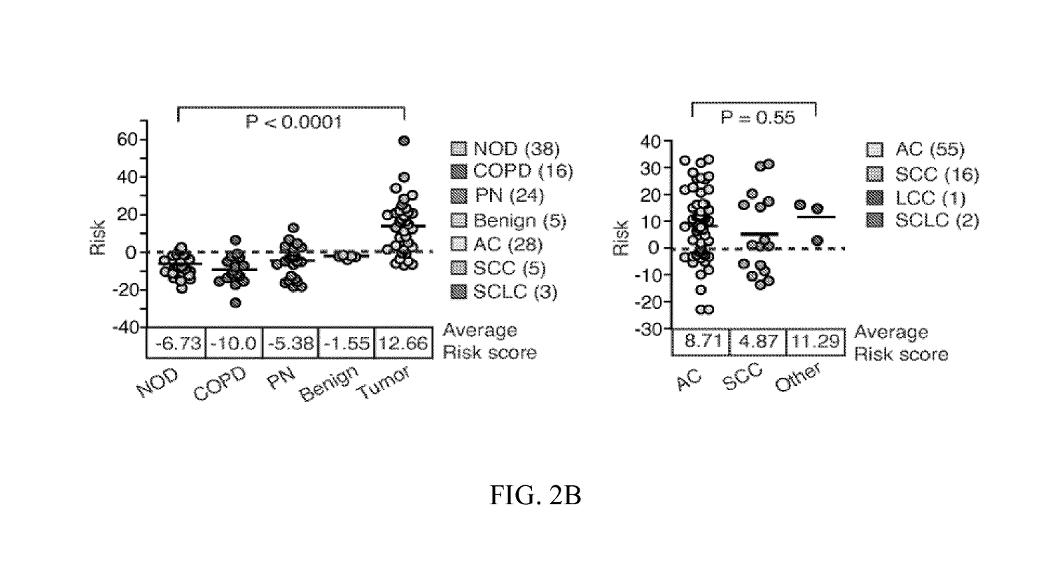

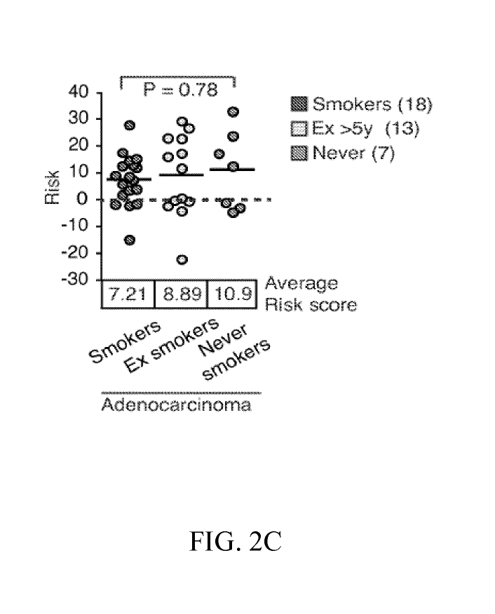

The miR-Test was then validated in an independent “Validation Set” of 1008 subjects enrolled in the COSMOS trial (FIG. 1, Table 1). In this set, the test displayed an AUC (area under curve) of 0.85 and an accuracy (ACC), sensitivity (SE) and specificity (SP) of 75%, 78% and 75%, respectively, when a risk score of 0 was used as cutoff (FIG. 2A, Table 5).

Next, a simulation of a “clinical” setting was attempted, in which the miRNA-Test would be used as a triage screening to identify individuals who should subsequently undergo LDCT. The Validation Set was stratified into three risk categories: high, intermediate, and low (see Example 1). By grouping together the high and intermediate risk categories, the sensitivity of the test increased to 86% (31 of 36 tumors, Table 5). Analysis of individuals in the low risk category (533 of 1008 (53%) including 5 tumors; Table 5), who would not be required to undergo LDCT, revealed a negative predictive value (NPV) of >99% for the miR-Test. The fact that 5 of 36 LDCT-detected tumors were classified as low risk (false negatives) by the miR-Test could reflect an intrinsic limitation of the test in its present form. However, in the case of the false negatives, we did not observe any death event out of the 15 deaths registered in all individuals screened (Validation and Clinical Sets). In addition, the death rate per 1000 patients/year was 0, 51, and 71, in the low, intermediate and high-risk categories, respectively. While the relatively low number of deaths in our cohort limits the statistical power of these results, it is plausible that tumors missed by the miR-Test may be rather indolent or even represent overdiagnosis by LDCT. Notably, however, none of the individuals in the Validation Set who were assigned a low risk score, either in the two- or three-category stratification, died of lung cancer in the follow-up period (>30 months) (Table 5).

Table 5 shows the performance of the miR-Test in various tests. The number of individuals assigned to difference miR-Test categories is reported. In brackets, percentage out of total. Tumor stage was defined based on the TNM Classification of Malignant Tumors published by the International Union Against Cancer (UICC), 7^{th} edition. ^{§}miR-Test performance using two-category stratification Pos: ≧0; Neg: <0. In Validation Set, sensitivity (SE) was 78% and specificity (SP) was 75%. In Specificity Set, SP was 87%. In Clinical Set, SE was 70%. * miR-Test performance using three-category stratification: High: ≧5; Intermediate: <5 and ≧−5; Low: <−5.

### Example 3: Analysis of the Performance of the miR-Test in Various Clinically Relevant Settings

A series of experiments to analyze the performance of the miR-Test in various clinically relevant settings was performed next. To assess the ability of the miR-Test to distinguish between non-malignant lung disease (NMD) and lung cancer, a “Specificity Set” was assembled selecting individuals from the COSMOS study (FIG. 1, Table 1) suffering from chronic obstructive pulmonary disease (COPD), benign pulmonary nodules or pneumonia (see Methods). In this set, 11 out of 83 (13%) individuals were assigned a positive score by the miR-Test when the two-category stratification was applied (FIG. 2B, left panel; Table 5). Of these, only three (4%) were classified as high-risk, when the three-category stratification was applied (Table 5). Importantly, 5 of 5 patients operated with a benign tumor (surgery false positives), were assigned a negative score by the miR-Test (FIG. 2B). Thus, the miR-Test displayed a high specificity (87%) in a set of individuals with NMD, further corroborating its reliability.

The miR-Test was then applied to a third independent set, the “Clinical Set”, composed of patients who were diagnosed with lung tumors outside of the COSMOS trial (FIG. 1, see Example 1). This analysis allowed to evaluate the performance of the test in an unselected population harboring more advanced lung cancers (Stage II-III), which are normally under-represented among screen-detected tumors (Table 1). The performance of the miR-Test in the Clinical Set was comparable to that in the Validation Set (SE, 70%; Table 5). Moreover, no major differences in performance were observed among the different tumor stages (Stage I, SE 69%; Stage II-III, SE 72%; Table 5) and subtypes (FIG. 2B) in the Clinical Set. In addition, in adenocarcinoma patients from the Clinical Set, no significant differences were observed among the risk scores of smokers, ex-smokers (>5 years) and never smokers (P=0.78; FIG. 2C).

Finally, serum samples of a group of stage I, non-small cell lung cancer (NSCLC) patients before and after surgery were analyzed, to evaluate miR-Test tumor specificity (see Example 1). At one month post-surgery, there was no significant overall decrease in the miR-Test risk scores (p=0.39; FIG. 3). In some patients the risk increased in the absence of any residual disease, possible to long-term stability of serum miRNAs and/or a release of miRNAs during surgery. Notably, at 5 months post-surgery there was a significant decrease in most patients (p=0.017, FIG. 3). In two patients, whose sera were available at 12 months post-surgery, the miR-Test risk score continued to decrease with respect to the 5 months risk score and yielded a negative test result in both cases (FIG. 3).

### Example 4: Origin of Circulating miR-Test miRNAs in Lung Cancer Patients

An interesting biological question pertains to the origin of circulating miRNAs and of their fluctuations in cancer patients. miRNA might be present in the serum due to their passive release from apoptotic cells, or active release from in microvesicles or in complex with miRNA-binding proteins, which protect them from degradation.

The expression of circulating miRNAs in the NCI-60 tumor cells dataset was analyzed. While such an approach has obvious caveats (e.g., expressed miRNAs are not necessarily secreted, and even if they are, their levels of expression might not reflect their levels of secretion), it was a feasible initial step which could direct further analyses.

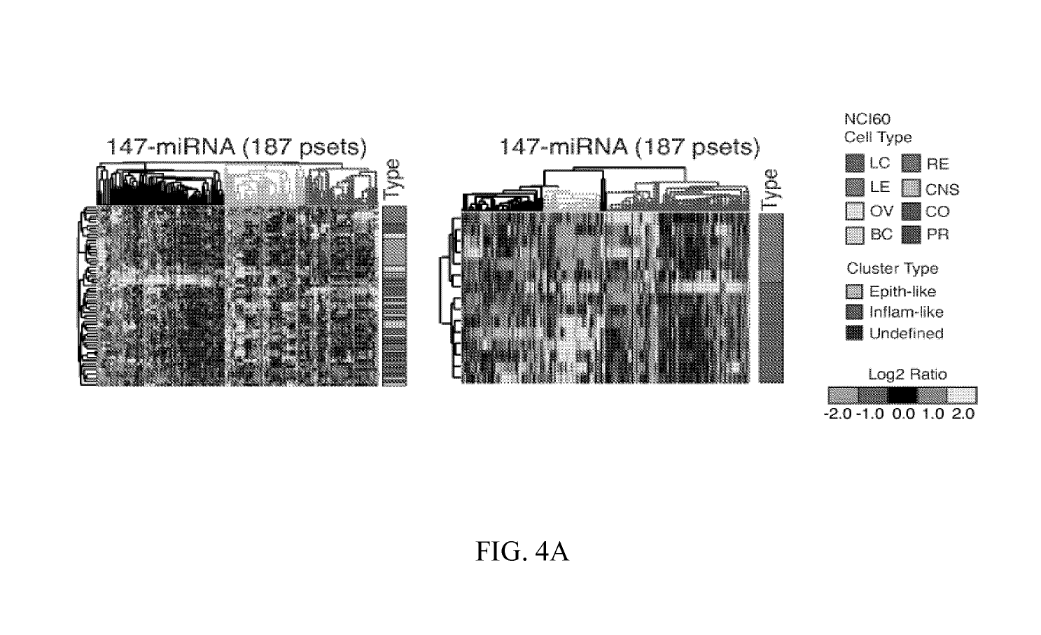

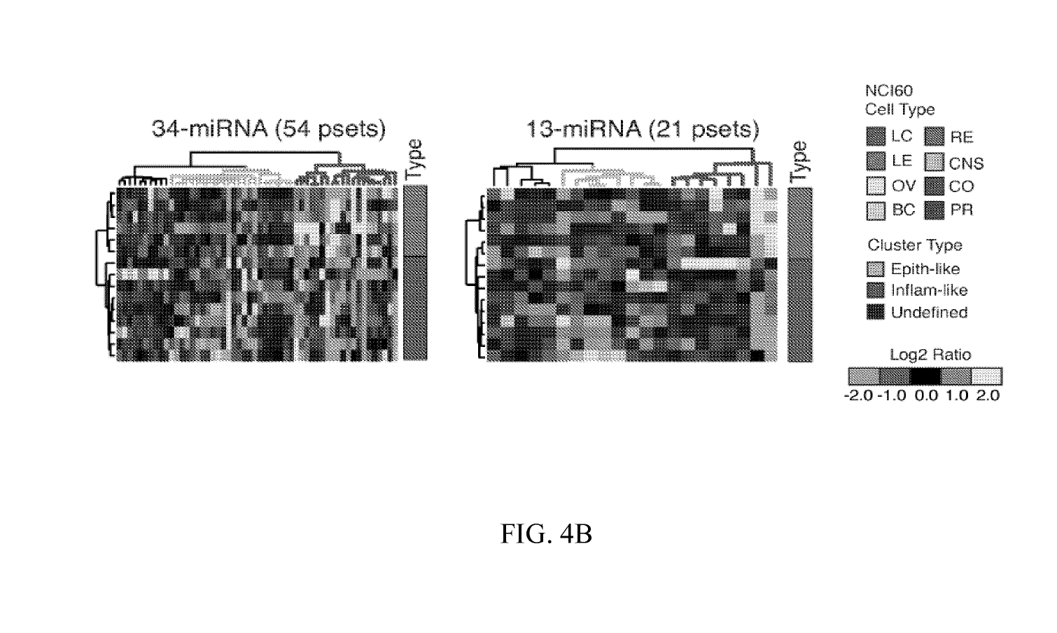

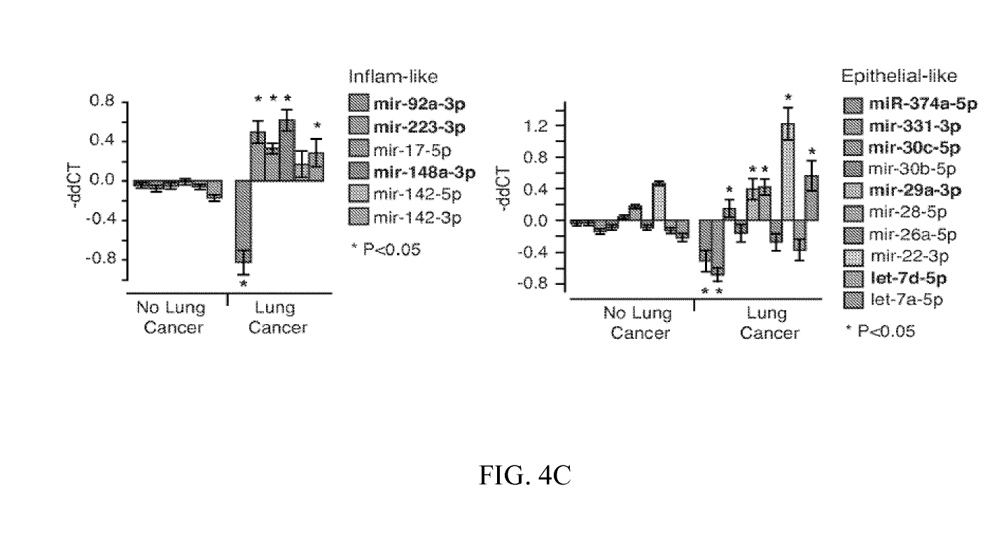

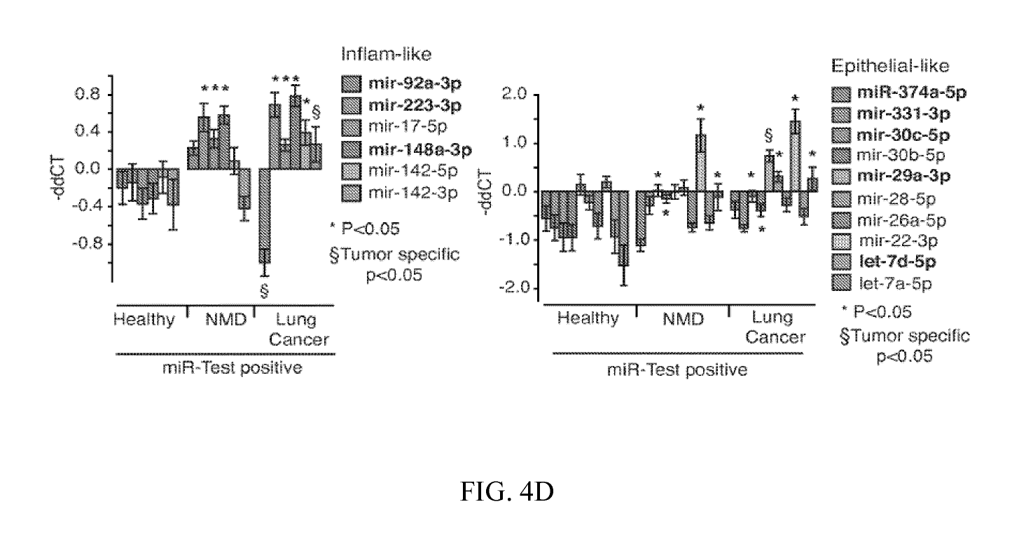

Initially, an unsupervised clustering analysis of all miRNAs that can be reliably detected in serum (147-miRNA, see Bianchi et al.) was performed. This yielded three main groups: i) miRNAs preferentially expressed in lines of hematopoietic origin, which we named the “inflammatory-like” cluster; ii) miRNAs preferentially expressed in lines of epithelial origin, which we named the “epithelial-like” cluster; iii) miRNAs without a clear differential pattern of expression (FIG. 4A). When the analysis was restricted to lung cancer and leukemic cell lines, the three-cluster structure was retained and more evident, regardless of whether the dataset was interrogated with the 147-miRNA signature (FIG. 4A, right panel) or with the 34- and 13-miRNA signatures (FIG. 4B). Statistical analyses confirmed a significant regulation of several miRNAs belonging to the 34- and 13-miRNA signatures in lung cancer or leukemic cell lines (16 and 8 of the 34- and 13-miRNA signatures, respectively; Table 6).

The above findings suggest a dual origin for the miRNAs of our signatures. Thus, the impact of the two components on the performance of the signature was investigated. First, it was established that both components are needed to maintain a good compromise between sensitivity and specificity (Table 7). Next, the levels of the epithelial- and inflammatory-like miRNAs, present in our signatures, in sera from individuals in the Validation Set were analyzed. The “inflammatory-like” miRNAs displayed a quasi-stereotypical behavior, with 5 of 6 miRNAs showing an increase in cancer patients vs. healthy individuals (FIG. 4C). The “epithelial-like” cluster, conversely, displayed a more heterogeneous behavior (FIG. 4C).

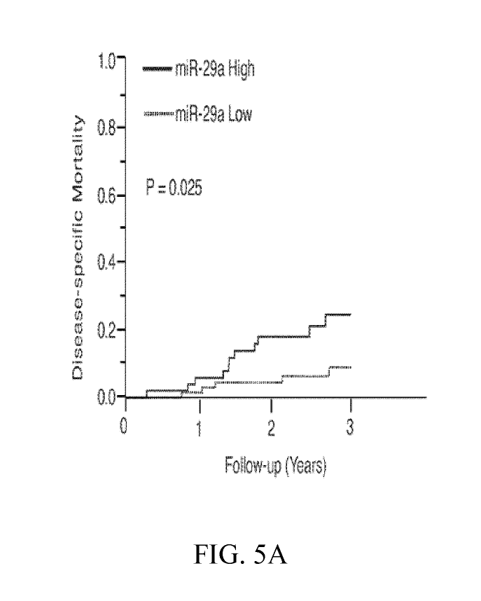

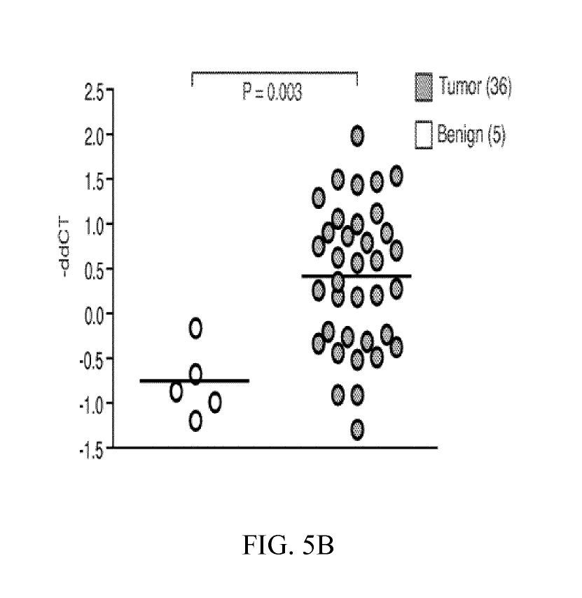

Finally, the expression of the epithelial- and inflammatory-like miRNAs in miR-Test-positive individuals in the Validation and Specificity Sets (FIG. 4D) was analyzed. This analysis unveiled two interesting features: i) a substantial fraction of the “inflammatory-like” miRNAs were increased in the serum of NMD patients (false positives), similarly to lung cancer patients, which is consistent with the presence of chronic or severe lung inflammation (FIG. 4D); ii) in the “epithelial-like” cluster, we found that miR-29a was significantly increased in patients with lung cancer (true positives), but not in patients with NMD, relative to healthy individuals (p=0.008; FIG. 4D), thereby representing a true tumor-specific miRNA. Accordingly, increased serum quantities of miR-29a correlated with an adverse prognosis (p=0.025; FIG. 5A). In addition, patients with benign tumors (surgery false positives) had lower amounts of miR-29a (p=0.003) in the serum compared to lung cancer patients (FIG. 5B).

Significantly regulated miRNAs of the 34-miRNA (or 13-miRNA) signature in the NCI-60 cell line panel, restricted to lung cancer (LC) and leukemic cell lines (LE) are shown in Table 6. Gene expression data were centered on their means (miRNA ID, microRNA OSU V3 chip relative pre-miRNA ID; Accession, miRBASE accession number; miRNA, mature miRNA name; in bold, mature miRNAs (N=16) belonging to the original 34-miRNA signature; miR-32 was excluded from the analysis because of its presence in both clusters; Cluster Type, identification of miRNA based on clustering analysis as described in the main text). P-values were calculated by the two-tailed t-test.

The sensitivity (SEN) and specificity (SPE) of the complete 13-miRNA miR-Test, and the miR-Test without the inflammatory-like (Epithelial-like) or epithelial-like (Inflam-like) components, in the Validation set (N=1008) is shown in Table 7. A risk score of 0 was used as cutoff to assign positive and negative test results.

TABLE 7
Evaluation of the diagnostic power of the “epithelial-like”
and “inflammatory”.

| | SEN | SPI |
|---|---|---|
| | % | % |
| miR-Test | 78% | 75% |
| Epithelial-like | 86% | 54% |
| Inflam-like | 69% | 84% |

### EQUIVALENTS

The details of one or more embodiments of the disclosure are set forth in the accompanying description above. Any methods and materials similar or equivalent to those described herein can be used in the practice or testing of the present methods and materials now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

### REFERENCES

1. Siegel R, Naishadham D, Jemal A. Cancer statistics, 2013. CA: a cancer journal for clinicians 2013; 63(1):11-30.
2. Henschke C I, Yankelevitz D F, Libby D M, et al. Survival of patients with stage I lung cancer detected on CT screening. The New England journal of medicine 2006; 355(17):1763-71.
3. Aberle D R, Adams A M, Berg C D, et al. Reduced lung-cancer mortality with low-dose computed tomographic screening. The New England journal of medicine 2011; 365(5):395-409.
4. Goulart B H, Bensink M E, Mummy D G, et al. Lung cancer screening with low-dose computed tomography: costs, national expenditures, and cost-effectiveness. Journal of the National Comprehensive Cancer Network: JNCCN 2012; 10(2):267-75.
5. Krol J, Loedige I, Filipowicz W. The widespread regulation of microRNA biogenesis, function and decay. Nature reviews. Genetics 2010; 11(9):597-610.
6. Yendamuri S, Kratzke R. MicroRNA biomarkers in lung cancer: MiRacle or quagMiRe? Translational research: the journal of laboratory and clinical medicine 2011; 157(4):209-15.
7. Schwarzenbach H, Hoon O S, Pantel K. Cell-free nucleic acids as biomarkers in cancer patients. Nature reviews. Cancer 2011; 11(6):426-37.
8. Shen J, Todd N W, Zhang H, et al. Plasma microRNAs as potential biomarkers for non-small-cell lung cancer. Lab Invest 2011; 91(4):579-87.
9. Hu Z, Chen X, Zhao Y, et al. Serum microRNA signatures identified in a genome-wide serum microRNA expression profiling predict survival of non-small-cell lung cancer. J Clin Oncol 2010; 28(10):1721-6.
10. Chen X, Ba Y, Ma L, et al. Characterization of microRNAs in serum: a novel class of biomarkers for diagnosis of cancer and other diseases. Cell Res 2008; 18(10):997-1006.
11. Bianchi F, Nicassio F, Marzi M, et al. A serum circulating miRNA diagnostic test to identify asymptomatic high-risk individuals with early stage lung cancer. EMBO molecular medicine 2011; 3(8):495-503.
12. Boeri M, Verri C, Conte D, et al. MicroRNA signatures in tissues and plasma predict development and prognosis of computed tomography detected lung cancer. Proceedings of the National Academy of Sciences of the United States of America 2011; 108(9):3713-8.
13. Kosaka N, Iguchi H, Yoshioka Y, et al. Secretory mechanisms and intercellular transfer of microRNAs in living cells. J Biol Chem 2010; 285(23):17442-52.
14. Skog J, Wurdinger T, van Rijn S, et al. Glioblastoma microvesicles transport RNA and proteins that promote tumour growth and provide diagnostic biomarkers. Nat Cell Biol 2008; 10(12):1470-6.
15. Valadi H, Ekstrom K, Bossios A, et al. Exosome-mediated transfer of mRNAs and microRNAs is a novel mechanism of genetic exchange between cells. Nat Cell Biol 2007; 9(6):654-9.
16. Vickers K C, Palmisano B T, Shoucri B M, et al. MicroRNAs are transported in plasma and delivered to recipient cells by high-density lipoproteins. Nat Cell Biol 2011; 13(4):423-33.
17. Arroyo J D, Chevillet J R, Kroh E M, et al. Argonaute2 complexes carry a population of circulating microRNAs independent of vesicles in human plasma. Proc Natl Acad Sci USA 2011; 108(12):5003-8.
18. Sozzi G, Boeri M, Rossi M, et al. Clinical utility of a plasma-based miRNA signature classifier within computed tomography lung cancer screening: a correlative MILD trial study. Journal of clinical oncology: official journal of the American Society of Clinical Oncology 2014; 32(8):768-73.
19. Veronesi G, Bellomi M, Mulshine J L, et al. Lung cancer screening with low-dose computed tomography: a non-invasive diagnostic protocol for baseline lung nodules. Lung cancer 2008; 61(3):340-9.
20. Hunter M P, Ismail N, Zhang X, et al. Detection of microRNA expression in human peripheral blood microvesicles. PloS one 2008; 3(11):e3694.
21. Rabinowits G, Gercel-Taylor C, Day J M, et al. Exosomal microRNA: a diagnostic marker for lung cancer. Clin Lung Cancer 2009; 10(1):42-6.
22. Turchinovich A, Weiz L, Langheinz A, et al. Characterization of extracellular circulating microRNA. Nucleic acids research 2011; 39(16):7223-33.
23. Wang K, Zhang S, Weber J, et al. Export of microRNAs and microRNA-protective protein by mammalian cells. Nucleic acids research 2010; 38(20):7248-59.
24. Turchinovich A, Weiz L, Burwinkel B. Extracellular miRNAs: the mystery of their origin and function. Trends in biochemical sciences 2012; 37(11):460-5.
25. Blower P E, Verducci J S, Lin S, et al. MicroRNA expression profiles for the NCI-60 cancer cell panel. Molecular cancer therapeutics 2007; 6(5):1483-91.
26. Henschke C I, McCauley Dl, Yankelevitz D F, et al. Early Lung Cancer Action Project: overall design and findings from baseline screening. Lancet 1999; 354(9173):99-105.
27. Patz E F, Jr., Pinsky P, Gatsonis C, et al. Overdiagnosis in low-dose computed tomography screening for lung cancer. JAMA internal medicine 2014; 174(2):269-74.
28. Leidner R S, Li L, Thompson C L. Dampening enthusiasm for circulating microRNA in breast cancer. PloS one 2013; 8(3):e57841.
29. Ein-Dor L, Kela I, Getz G, et al. Outcome signature genes in breast cancer: is there a unique set? Bioinformatics 2005; 21(2):171-8.
30. Reinhold W C, Sunshine M, Liu H, et al. CellMiner: a web-based suite of genomic and pharmacologic tools to explore transcript and drug patterns in the NCI-60 cell line set. Cancer research 2012; 72(14):3499-511.

1. A method of diagnosing lung cancer in a subject in need thereof comprising
(a) detecting in a biological sample from the subject a decrease in the abundance of each of hsa-mir-92a-3p, hsa-mir-30b-5p, hsa-mir-191-5p, hsa-mir-484, hsa-mir-328-3p, hsa-mir-30c-5p, hsa-mir-374a-5p, hsa-mir-7d-5p, and hsa-mir-331-3p compared to a control abundance value corresponding to each of hsa-mir-92a-3p, hsa-mir-30b-5p, hsa-mir-191-5p, hsa-mir-484, has-mir-328-3p, hsa-mir-30c-5p, hsa-mir-374a-5p, hsa-mir-7d-5p, and hsa-mir-331-3p from a control sample;
(b) detecting in the biological sample an increase in the abundance of each of hsa-mir-29a-3p, hsa-mir-148a-3p, hsa-mir-223-3p, and hsa-mir-140-5p compared to a control abundance value corresponding to each of hsa-mir-29a-3p, hsa-mir-148a-3p, hsa-mir-223-3p, and hsa-mir-140-5p from a control sample; and
(c) diagnosing the subject with lung cancer, when a decreased abundance in each of the miRNA in (a) is detected and an increased abundance of each of the miRNA in (b) is detected.

2. The method of claim 1, wherein the biological sample comprises a biological fluid.

3. The method of claim 2, wherein the biological fluid is saliva, urine, blood, or lymph fluid.

4. The method of claim 1, wherein biological sample comprises blood, whole blood, blood plasma and/or blood serum.

5. The method of claim 1, wherein the biological sample comprises blood serum.

6. The method of claim 1, wherein the decrease is a statistically significant difference from the control value, or the increase is a statistically significant difference from the control value, or both.

7. The method of claim 6, wherein a statistically significant difference is characterized by a p-value of less than 0.05.

8. The method of claim 7, wherein a statistically significant difference is characterized by a p-value of less than 0.001.

9. The method of claim 8, wherein a statistically significant difference is characterized by a p-value of less than 0.0001.

10. (canceled)

11. The method of claim 1, further comprising the step of calculating a risk score.

12. The method of claim 11, wherein the risk score is a product of
(a) a fold decrease and a weight coefficient, or
(b) a fold increase and a weight coefficient,
wherein the weight coefficient is determined by a diagonal linear discriminant analysis (DLDA).

13. The method of claim 1, wherein the control value for each miRNA is determined by a method comprising detecting in a control biological sample from a normal subject an abundance of each of hsa-mir-92a-3p, hsa-mir-30b-5p, hsa-mir-191-5p, hsa-mir-484, hsa-mir-328-3p, has-mir-30c-5p, hsa-mir-374a-5p, hsa-mir-7d-5p, hsa-mir-331-3p, hsa-mir-29a-3p, hsa-mir-148a-3p, hsa-mir-223-3p, and hsa-mir-140-5p.

14. The method of claim 13, wherein the normal subject does not have cancer.

15. The method of claim 13, wherein the normal subject does not have lung cancer.

16. The method of claim 13, wherein the control biological sample comprises a biological fluid.

17. The method of claim 16, wherein the control biological fluid is saliva, urine, blood, or lymph fluid.

18. The method of claim 13, wherein the control biological sample comprises blood, whole blood, blood plasma and/or blood serum.

19. The method of claim 13, wherein the control biological sample comprises blood serum.

20. The method of claim 13, wherein the control biological sample comprises blood, whole blood, blood plasma and/or blood serum obtained from at least two normal subjects.

21. The method of claim 19, wherein the control biological sample comprises blood serum obtained from at least two normal subjects.

22-23. (canceled)

24. The method of claim 1, wherein the subject has stage I lung cancer.

25. The method of claim 1, wherein the subject has stage II lung cancer.

26-29. (canceled)

30. The method of claim 1, further comprising the step of performing low-dose computed tomography (LDCT) or referring the subject for LDCT if the subject is diagnosed with lung cancer.

31. The method of claim 1, further comprising the step of providing a treatment or referring the subject for treatment if the subject is diagnosed with lung cancer.

32. The method of claim 1, wherein the detecting step of (a) and/or (b) comprises
transcribing each of the miRNA in (a) and/or (b) using at least one human miRNA stem-loop primer specific for at least one miRNA of (a) or (b) to generate a complementary DNA (cDNA) corresponding to each of the miRNA,
amplifying each of the cDNA, and determining a relative abundance of each of the cDNA compared to at least one housekeeping miRNA.

33. The method of claim 32, wherein the at least one human miRNA stem-loop primer specific for at least one miRNA and the at least one miRNA hybridize to form at least one duplex.

34. The method of claim 32, wherein the amplifying step comprises performing quantitative real-time polymerase chain reaction (qRT-PCR).

35. The method of claim 32, wherein the at least one housekeeping miRNA comprise miR-197, miR-19b, miR-24, miR-146, miR-15b, or miR-19a.

36. The method of claim 33, wherein the housekeeping miRNA are miR-197, miR-19b, miR-24, miR-146, miR-15b, and miR-19a.

37. The method of claim 33, wherein the relative abundance of each of the cDNA is determined by adding a scaling factor to the raw cycle threshold (CT) of each of the cDNA to generate a normalized (CT) value.

38. The method of claim 37, wherein the scaling factor is determined by determining a mean cycle threshold of the at least two housekeeping miRNA selected from the group consisting of miR-197, miR-19b, miR-24, miR-146, miR-15b, and miR-19a and subtracting the mean CT from a constant value (K).

39. The method of claim 37, wherein the constant value (K) equals 21.646.

40. The method of claim 37, wherein the normalized CT value is used to determine a weight coefficient.

41. The method of claim 1, further comprising the step of determining a risk score for the subject.

42. The method of claim 41, wherein the risk score is calculated according to: RS=−(Σᵢwᵢxᵢ−THRES),
wherein i corresponds to each of the miRNAs of (a) and (b), respectively, and
wherein THRES=−261.779, wᵢ is the weight coefficient of the i^{th} miRNA, and xᵢ is the expression value of the i^{th} miRNA.

43. The method of claim 42, wherein the expression value of the i^{th} miRNA is the raw CT of the i^{th} miRNA.

44. The method of claim 41, wherein the risk score is greater than or equal to 5, indicating that the subject has a high risk of developing cancer.

45. The method of claim 41, wherein the risk score is less than 5 and greater than or equal to −5, indicating that the subject has an intermediate risk of developing cancer.

46. The method of claim 41, wherein the risk score is less than −5, indicating that the subject has a low high risk of developing cancer.
